(19) United States
(12) Patent Application Publication
(10) Pub. No.: US 2025/0170417 A1
(43) Pub. Date: May 29, 2025

(54) SLEEP INDUCTION SYSTEM USING PHOTOBIOMODULATION ACCORDING TO TRANSCRANIAL NEAR-INFRARED IRRADIATION

(71) Applicant: TEDI MEDI CO., LTD., Gwangju (KR)

(72) Inventors: Jae Gwan KIM, Gwangju (KR); Tae KIM, Gwangju (KR)

(21) Appl. No.: 18/730,596

(22) PCT Filed: Jan. 19, 2023
(86) PCT No.: PCT/KR2023/000904

§ 371 Date: Jul. 19, 2024

(30) Foreign Application Priority Data
Jan. 21, 2022 (KR) 10-2022-0009402

(51) Int. Cl. A61N 5/06; A61M 21/02

(52) U.S. Cl. CPC A61N 5/0618; A61M 21/02; A61N 2005/0647; A61N 2005/0651; A61N 2005/0658

(57) ABSTRACT

Embodiments relate to a sleep induction system using photobiomodulation according to transcranial near-infrared irradiation. In the health and medical fields, the present system can be used as a method with minimal side effects, which can induce sleep by increasing sleep pressure, with a freedom from the problems of dependence on drug sleeping pills and long-term use.

### TECHNICAL FIELD

The present invention relates to a sleep induction system using photobiomodulation (PBM) with transcranial near-infrared irradiation, which can increase sleep induction by increasing sleep pressure by transcranial photobiomodulation using near-infrared light.

### BACKGROUND ART

Sleep disorders are common among modern people, and are prevalent conditions that over 20% of the population have experienced or are suffering from the disease. Sleep disorders refer to the inability to achieve healthy sleep, the inability to maintain wakening during the day despite sufficient sleep, or experiencing difficulties in sleeping or staying awake due to disrupted sleep rhythms. Mental issues, such as excessive stress, anxiety, tension, and fear, often cause sleep disorders. Sleep disorders can lead to various personal and social problems, such as learning disabilities, decreased efficiency, traffic accidents, various incidents, emotional disorders, social adjustment disorders, marital dissatisfaction, and industrial accidents. Moreover, if sleep disorders are not properly treated, it may exacerbate existing medical, neurological, and psychiatric conditions, delay recovery of the existing conditions, and even lead to severe diseases such as myocardial infarction and stroke. To treat the sleep disorders, chemical sleep aids which reversibly suppress the central nervous system to induce and maintain sleep are used. Traditional sleep aids, similar to anesthetics, have a central depressant effect where a small dose can cause sedation, a moderate dose can induce sleep, but a high dose can result in coma, paralysis, and respiratory depression. Barbiturate medications easily develop tolerance and dependency due to the low safety, and if stopping taking the medications after long-term use, people may suffer from sleep disorders caused by nightmares.

Recently, benzodiazepine medications, which have fewer harmful effects and possess anxiolytic properties, are being used. The medications, including benzodiazepines, bind to the GABA_{A} receptors and perform pharmacological actions. When drugs or adjuvants bind to the benzodiazepine, it enhances the affinity of the GABA_{A} receptors to GABA, and increase the influx of chloride ions into cells, resulting in anxiety relief, improvement of spasms, sedation, and induction and improvement of sleep. GABA_{A} receptors are pentameric protein that form membrane ion channels, are closely associated with the regulation of sedation, sleep, anxiety, muscle tension, spasms, and amnesia, thereby facilitating the action of GABA (gamma-aminobutyric acid). Long-term use of the medications can lead to side effects such as tolerance and dependency, may cause symptoms like muscle relaxation, amnesia, lethargy, comatose state, coronary vasodilation, and myocardial blockage, and specifically, may cause congenital abnormalities if used by pregnant women.

As another method for treating sleep disorders, there is a method that increases homeostatic sleep pressure through an increase of adenosine to enhance sleep induction. Sleep pressure refers to the tendency to sleep that increases in proportion to waking hours, and in terms of brainwaves, sleep pressure can be measured by delta waves during sleep or theta waves during wakening. Normally, by the evening, sufficient sleep pressure is accumulated, thus allowing natural sleep induction. However, if sleep pressure is low in the evening or if wakening exceeds sleep pressure, sleep problems may occur. Although adenosine elevation can be induced through sleep deprivation, it is practically challenging. Alternatively, the intake of alcohol may induce an increase of adenosine in the extracellular space by blocking ENTI transporters moving adenosine into cells, but may lead to reduced sleep quality due to the side effects of alcohol. Recently, substances that enhance the function of the adenosine A2A receptor, rather than increasing adenosine, have been discovered and shown to have sleep-enhancing effects. However, since adenosine receptors are widespread throughout the body, there are concerns about side effects on bodily functions.

The transcranial near-infrared light stimulator is a medical device that non-invasively applies light to the transcranial area using near-infrared light sources to stimulate the brain, and is used for the rehabilitation of traumatic brain injuries, dementia, and Alzheimer's by activating the mitochondria in the brain. The transcranial near-infrared light stimulator includes a plurality of near-infrared light sources arranged in series and parallel, and generates direct current voltage and current corresponding to each of the near-infrared light sources to provide light stimulation. The transcranial near-infrared light stimulator promotes the production of adenosine triphosphate (ATP), a vital energy source for living bodies by enhancement of the activity of mitochondria within brain neurons, to effectively suppress inflammation generated within the brain. As a result, it is reported that the transcranial near-infrared light stimulator is effective not only for the rehabilitation of brain diseases, such as strokes, but also for treatment of neurological disorders like sleep disorders, depression, epilepsy, dementia, Parkinson's, tic disorders, tinnitus, addiction, chronic pain, and insomnia.

Accordingly, the inventors of the present invention have developed a sleep induction system using photobiomodulation with transcranial near-infrared irradiation, which can elevate homeostatic sleep pressure through an increase of adenosine, and have confirmed that sleep pressure can be increased prior to sleep onset by the transcranial photobiomodulation (PBM) using near-infrared light to induce sleep.

### DISCLOSURE

### Technical Problem

Accordingly, the present invention has been made in view of the above-mentioned problems occurring in the related art, and it is an object of the present invention to provide a sleep induction system using transcranial photobiomodulation (PBM) with transcranial near-infrared light.

### Technical Solution

To accomplish the above-mentioned objects, according to the present invention, there is provided a photobiomodulation (PBM) system for inducing sleep, including: a light source for light irradiation; and a control unit for adjusting the output of light.

In an embodiment, the light source irradiates light of near-infrared wavelength to the cranium.

In an embodiment, the near-infrared wavelength ranges from 650 nm to 1100 nm.

In an embodiment, the near-infrared wavelength is 850 nm.

In an embodiment, during light irradiation from the light source, mitochondria are activated to increase ATP, and the increased ATP is decomposed into adenosine, thereby increasing the amount of adenosine within the living body.

In an embodiment, the increased adenosine increases sleep pressure, thereby inducing a sleep enhancement effect.

In an embodiment, after the light irradiation from the light source, sleep pressure, which is expressed as a high level of wakening theta waves, is increased.

In an embodiment, the increase in sleep pressure creates a brain environment that induces an enhancement of delta waves during sleep.

In an embodiment, the light irradiation from the light source is performed prior to sleep onset.

In an embodiment, the control unit adjusts the light wavelength, irradiation intensity, and duration of irradiation.

In addition, in another aspect of the present invention, there is provided a sleep induction method using the PBM system.

### Advantageous Effect

According to an embodiment of the present invention, the photobiomodulation (PBM) system for sleep induction, which includes a light source unit for irradiation and a control unit for adjusting the output of light, can induce sleep by increasing sleep pressure, thereby being utilized to treat sleep disorders.

The effects of the present invention are not limited to those mentioned above and should be understood to include all possible effects inferable from the description or claims of the present invention.

### DESCRIPTION OF DRAWINGS

FIG. 1 depicts wakening time, NREM time, and REM time observed during and after PBM stimulation.

FIG. 2 shows averages of the wakening time, the NREM time, and the REM time, which are observed during PBM stimulation (Stim), after PBM stimulation (Post-stim), and during the total hours of the Stim and the Post-stim.

FIG. 3 shows analysis results of the number and the length of episodes during PBM stimulation (Stim), after PBM stimulation (Post-stim), and during the total hours of the Stim and the Post-stim.

FIG. 4 shows comparison results between a brainwave power spectrum in a wakening state and a brainwave power spectrum in a sleep state.

FIG. 5 shows the comparison average values of low-frequency delta waves during PBM stimulation (Stim), after PBM stimulation (Post-stim), and during the total hours of the Stim and the Post-stim relative to brainwaves observed during sleep (NREM).

FIG. 6 illustrates changes in the size of wakening theta waves (left) and sleep delta waves (right) observed during and after PBM stimulation.

FIG. 7 illustrates the correlation between the size of NREM delta waves observed before (left) and during (right) PBM application and the size of wakening theta waves.

FIG. 8 illustrates results of adenosine concentration measurements through frontal lobe microdialysis during three hours of PBM application, and during the first and second three hours after PBM application.

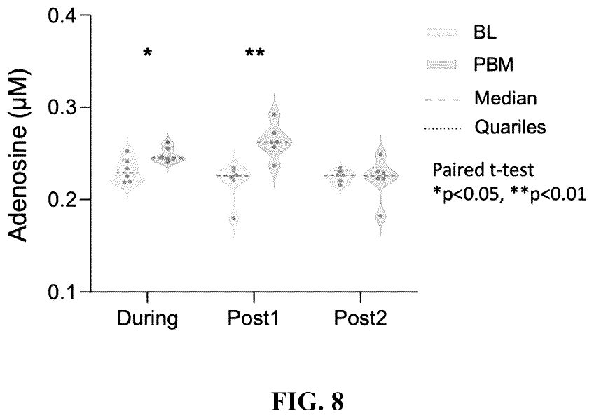

FIG. 9 is a structural diagram of a device to which a PBM system is applied for sleep induction.

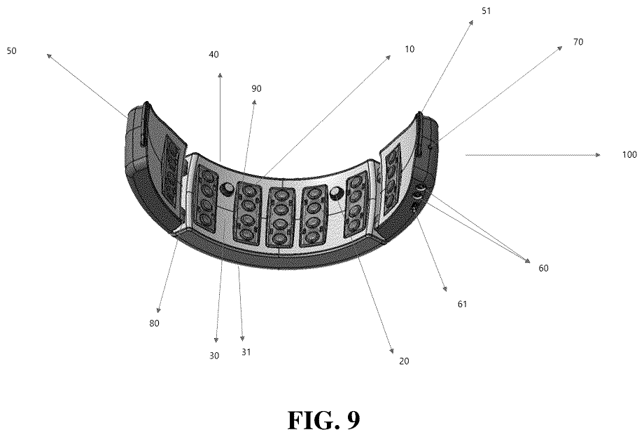

FIG. 10 illustrates a device according to an embodiment of the PBM system for sleep induction.

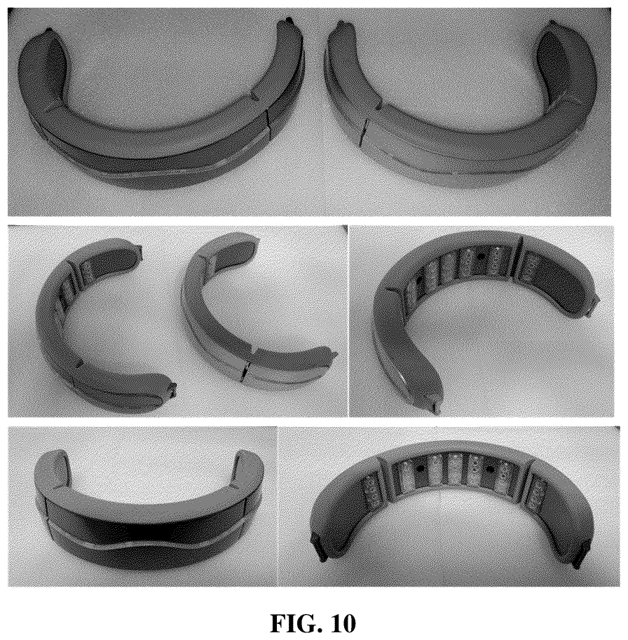

FIG. 11 is a schematic diagram illustrating a state in which a user wears the device to which the PBM system is applied for sleep induction.

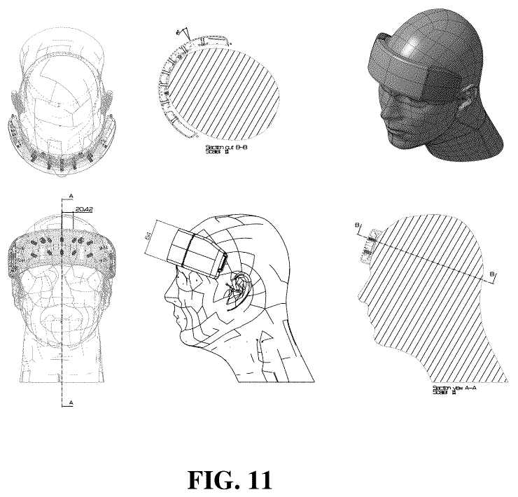

### MODE FOR INVENTION

Hereinafter, the present invention will be described in detail.

According to one aspect of the present invention, a photobiomodulation (PBM) system for sleep induction, which includes: a light source for irradiation; and a control unit for adjusting the output of light, is provided.

In the present invention, photobiomodulation (PBMT or PBM) is a method of irradiating relatively low intensity laser or light with power ranging from 1 to 500 mW in the wavelength range of 650 nm to 1,100 nm, and is based on the principle that specific molecules in a living system can absorb photons and trigger signaling pathways in response to light. PBM is known to be effective in wound healing promotion, pain relief, anti-inflammatory and anti-edematous effects, and neural regeneration, and is applied broadly in medical fields, for example, treatment of various traumatic and degenerative diseases. In the present invention, sleep induction can be achieved using the PBM method that irradiates light of specific wavelengths to the cranium.

According to an embodiment of the present invention, PBM for sleep induction can be realized to include the step of irradiating light to the cranium.

The near-infrared wavelength can range from 650 nm to 1,100 nm, and the penetration depth into the skull may vary depending on the wavelength.

In the present invention, transcranial PBM using near-infrared light activates Cytochrome c oxidase in the mitochondria within cells to increase ATP synthesis. When ATP is increased in the extracellular space, the increased ATP is either degraded into adenosine by 5′-ecto nucleotidase or into AMP by AMP deaminase. Thus, the present invention can increase adenosine levels in vivo through transcranial PBM using near-infrared light.

During irradiation by the light source, the activation of mitochondria increases ATP, and the increased ATP is then degraded into adenosine, thereby increasing the amount of adenosine within the living body.

The increased adenosine can enhance sleep pressure, thereby inducing sleep enhancement effects.

During irradiation by the light source, the increase in brain theta waves during wakening may be a result of increased sleep pressure, and such an increase in sleep pressure may lead to an increase in delta waves during sleep, creating a brain state for sleep onset and maintenance.

Specifically, the increase in the amount of adenosine due to light irradiation results in an increase in sleep pressure, which is expressed as a high level of theta waves, and the increase in sleep pressure induces an increase of delta waves during sleep, which is a representative index of deep sleep. Irradiation of near-infrared light for a predetermined period before bedtime facilitates sleep onset through the above-mentioned mechanism, and creates a brain environment that induces an increase of delta waves during sleep, and the brain environment is maintained for a certain period after the end of irradiation.

In the present invention, the term “sleep pressure” refers to an indicator that shows how much sleep is needed. Sleep pressure rises during awakening and gradually decreases during sleep.

In the present invention, the term “theta waves” refers to one type of brainwaves, and theta waves occur during sleep with a frequency of 4 to 8 cycles per second (or 4 to 8 Hz). “Delta waves” occur during deep sleep with a frequency of 0.5 to 4 cycles per second (or 0.5 to 4 Hz).

In the present invention, the term “fragmentation” refers to a state of unstable sleep characterized by waking up frequently, and “wakening” refers to a state of being awake, that is, not entering sleep.

The light irradiation of the light source unit may be performed between a time point prior to sleep onset and sleep onset time.

The control unit may regulate the light wavelength, irradiation intensity, and duration of irradiation.

Furthermore, according to the present invention, a method for inducing sleep using the PBM system can be provided.

In the present invention, the term nonrapid eye movement sleep (NREM) refers to a period of sleep where brainwaves of lower frequencies, such as delta waves, predominantly appear on an electroencephalogram, and means deep sleep. Rapid eye movement sleep (REM) refers to light sleep where theta waves predominantly appear on an electroencephalogram, and shows amplitudes similar to the period of wakening.

Hereinafter, the present invention will be described in more detail through embodiments and experimental examples. However, the following embodiments and experimental examples are provided for illustration purposes only, and the scope of the present invention is not limited thereto.

### Experimental Example

### Effectiveness Validation Through Animal Experiment

Optical fiber was fixed to the surface of the skull of a mouse and connected to an 850 nm light source, allowing free movement. Thereafter, a device for PBM was implanted to the skull of the mouse, and at the same time, electroencephalogram electrodes were fixed to the skull to record brainwaves.

PBM was performed on the mouse from the start of a light sleep period (7 AM) for three hours, and electroencephalogram recordings were conducted during PBM and for three hours after PBM, totaling 6 hours. Additionally, ground state electroencephalogram was measured and recorded from 7 AM to 1 PM the day before PBM for comparison.

### <Result Analysis>

### 1. Measurement of Sleep-Related Variables Through Near-Infrared PBM on the Skull Surface

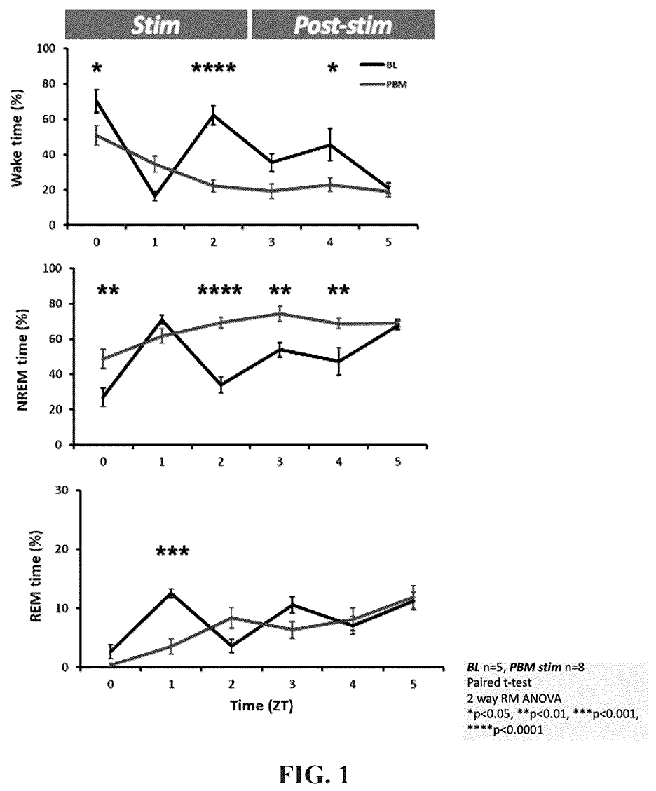

As illustrated in FIG. 1, wakening and sleep times for three hours during the near-infrared irradiation (Stim) to the skull of the mouse and for three hours after the near-infrared irradiation (Post-stim) were analyzed every hour, and as results of analysis, it was revealed that wakening was reduced but non-rapid eye movement sleep (NREM) was increased.

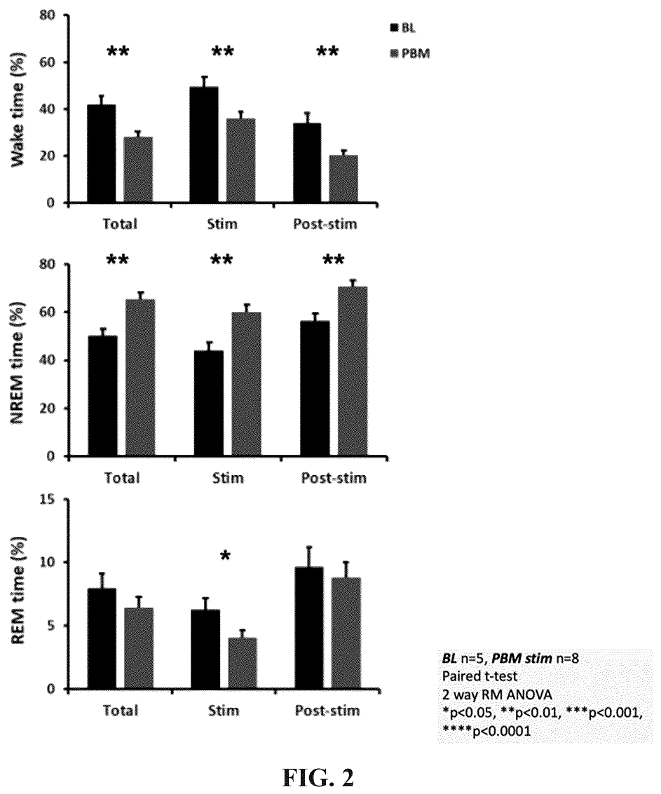

Furthermore, as illustrated in FIG. 2, as an integrated analysis of a total of six hours and per 3 hours (Stim and Post-stim), there were statistically significant changes that NREM time was increased and wakening time was decreased. However, REM sleep showed a significant decrease during near-infrared irradiation (Stim).

### 2. Episode Analysis for Understanding Sleep and Wakening Characteristics

The detailed characteristics of each sleep and wakening were identified through episode analysis. During the stimulation period (Stim), the number of wakening and NREM episodes increased, but their duration showed a decreasing trend. However, during the post-stimulation period (Post-stim), both the number and duration of episodes showed an increasing trend.

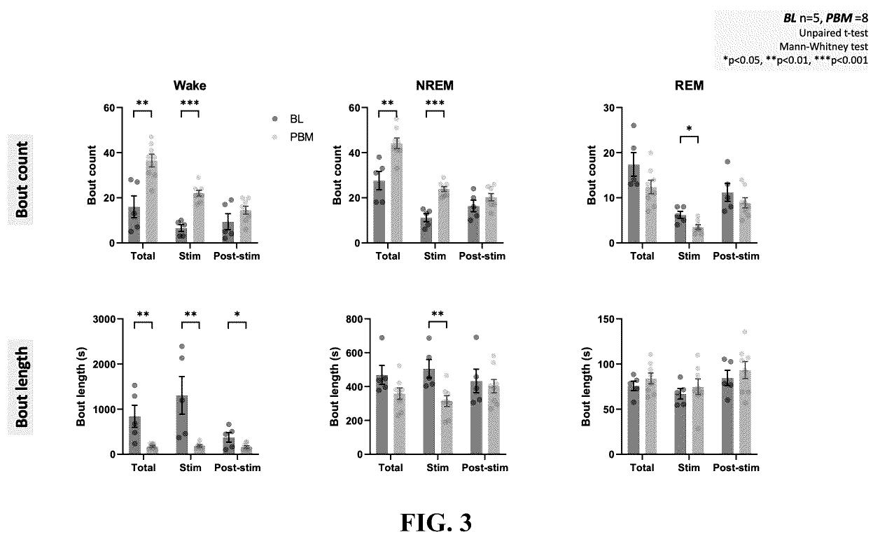

The results indicate that during stimulation, there was rapid transitioning of sleep and NREM episodes, that is, frequent fragmentation (FIG. 3).

As described above, referring to FIGS. 1 and 2, it was confirmed that PBM induced an increase in sleep, and also induced an increase in fragmentation. It showed that an increase of wakening and an increase of NREM sleep were showed together since the increase of ATP and the increase of adenosine occurred simultaneously.

### 3. Frequency Analysis of Brainwaves

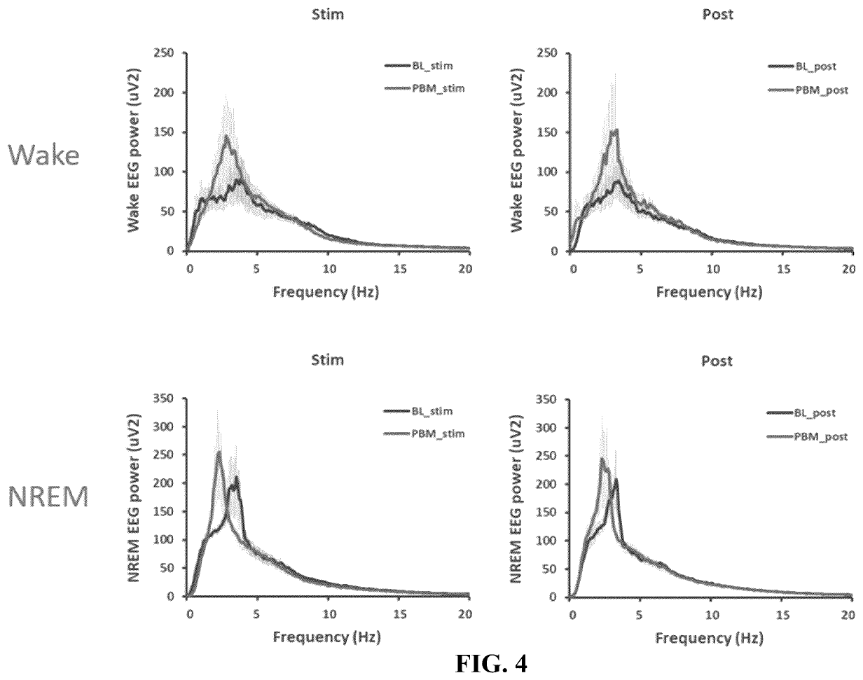

As illustrated in FIG. 4, through the analysis of brainwaves, it was confirmed that delta waves (0.5 to 4 Hz) during the near-infrared irradiation period and during the post-irradiation were increased, and it was indicated sleep propensity during wakening was increased. Specifically, low-frequency delta waves (0.5 to 2 Hz) during NREM sleep were increased compared to the ground state having no PBM. The NREM delta waves, especially in the low-frequency range, are the most crucial biomarker for sleep propensity. The sleep propensity during previous wakening was reflected to the NREM sleep brainwaves.

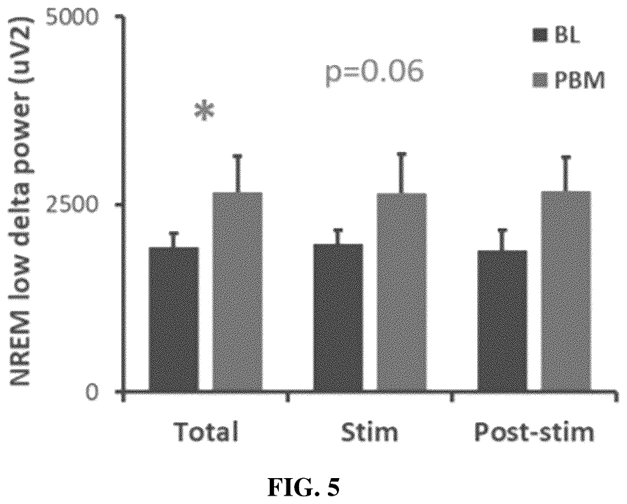

That is, as illustrated in FIG. 5, in the six-hour analysis, there was a statistically significant increase in NREM low-frequency delta waves, and when analyzed separately during stimulation and after stimulation, similar levels of increase tendency were confirmed.

### 4. Analysis of Sleep Pressure Indicators (Theta and Delta Waves)

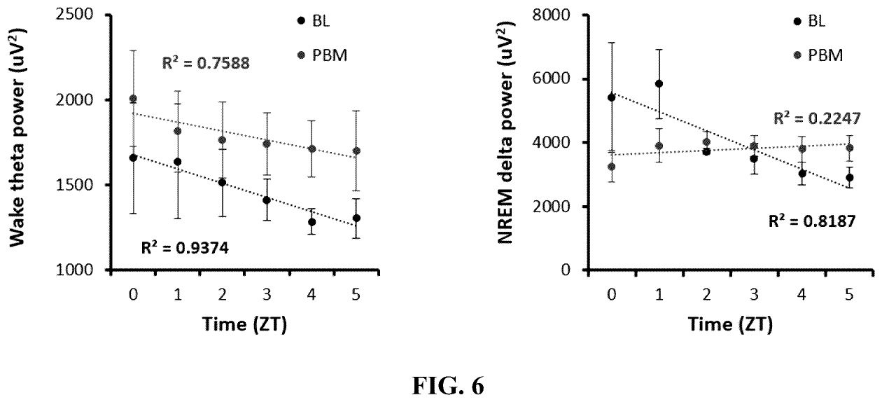

The two primary indicators of sleep pressure, theta waves during wakening and delta waves during sleep, were analyzed for three hours of near-infrared irradiation and for three hours of post-irradiation. As illustrated in FIG. 6, the two indicators tend to decrease as sleep progresses normally. In the baseline state without stimulation (black color), the normal decreasing tendency was apparent, but during PBM application (red color), the decrease in wakening theta waves was attenuated, and sleep delta waves were increased during sleep. The results indicate that during near-infrared irradiation, generation of adenosine, which contributes to sleep pressure at the beginning of sleep, is increased to induce a state conducive to sleep onset maintenance.

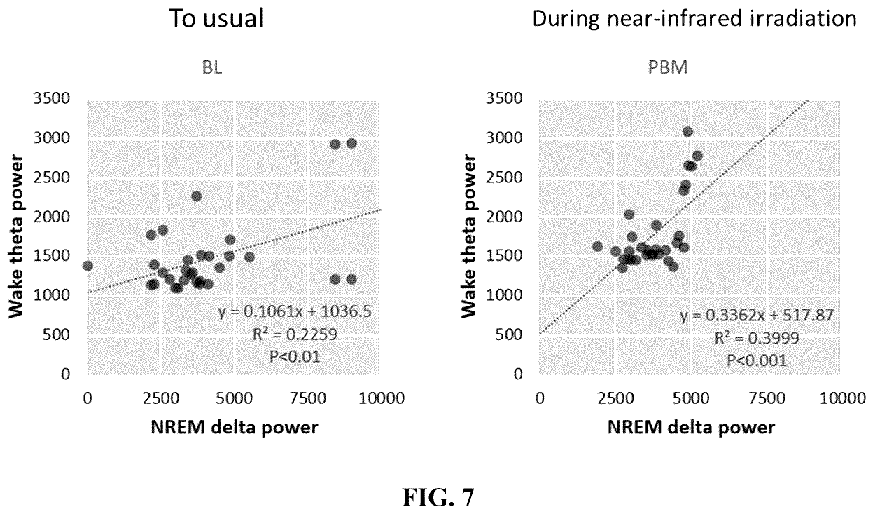

In general, NREM delta waves and wakening theta waves, which are the primary biomarkers of homeostatic sleep, show a positive correlation. As illustrated in FIG. 7, the positive correlation exhibited an increased slope during near-infrared irradiation compared to usual. It means that wakening theta waves were higher than sleep delta waves, that sleep pressure during wakening increased compared to usual, and that an environment within the brain was created to be conducive to an increase of subjective sleepiness and a faster sleep onset.

### 5. Measurement of Adenosine

A microdialysis probe was inserted into the prefrontal cortex to measure the concentration of adenosine within the brain, so, it was confirmed that high concentrations of adenosine were maintained during the three-hour Stim period and during the three-hour Post-stim.

Through the five analysis results, it was found that the change in brainwaves induced by PBM is continued four about three hours not only during the PBM irradiation period but also after the PBM irradiation, and that during the irradiation period, a mixed effect of wakening enhancement of ATP and sleep promotion of adenosine can be induced. Therefore, the effects of sleep induction can be obtained through the pure increase in sleep pressure when PBM is conducted prior to sleep onset and PBM application is stopped right before sleep onset.

The above description is only exemplary, and it will be understood by those skilled in the art that the invention may be embodied in other concrete forms without changing the technological scope and essential features. Therefore, the above-described embodiments should be considered only as examples in all aspects and not for purposes of limitation. For example, each component described as a single type may be realized in a distributed manner, and similarly, components that are described as being distributed may be realized in a coupled manner.

The scope of the present invention is defined by the appended claims, and encompasses all modifications or alterations derived from meanings, the scope and equivalents of the appended claims.

### SEQUENCE LIST TEXT

10: LED light source element
20: Electroencephalogram electrode
30: Internal chassis
31: External chassis
40: Frontal wear fixation unit
50: Lateral wear fixation unit
51: Strap hook
60: Connection terminal
61: Charging cable connection unit
70: Operation display unit
80: Lateral adjustment rotation unit
90: Light source protection unit
100: PBM system device for sleep induction

### NATIONAL R&D PROJECTS THAT SUPPORTED THIS INVENTION

[Assignment unique number] RS-2023-00303346
[Department name] Ministry of SMEs and Startups
[Research project name] TIPS (Tech Incubator Program for Startup) program
[Research Study Name] Development of near-infrared light-based wearable device for sleep improvement
[Research Management Professional Organization] Small and Medium Enterprise Technology Information Promotion Agency
[Contribution rate] 100%
[Main Research Organization] TEDI MEDI CO., LTD.
[Research period] 2023 Oct. 1˜2025 Sep. 30

1. A photobiomodulation (PBM) system for inducing sleep, comprising:
a light source for light irradiation; and
a control unit for adjusting the output of light.

2. The PBM system according to claim 1, wherein the light source irradiates light of near-infrared wavelength to the cranium.

3. The PBM system according to claim 2, wherein the near-infrared wavelength ranges from 650 nm to 1100 nm.

4. The PBM system according to claim 2, wherein the near-infrared wavelength is 850 nm.

5. The PBM system according to claim 1, wherein during light irradiation from the light source, mitochondria are activated to increase ATP, and the increased ATP is decomposed into adenosine, thereby increasing the amount of adenosine within the living body.

6. The PBM system according to claim 5, wherein the increased adenosine increases sleep pressure, thereby inducing a sleep enhancement effect.

7. The PBM system according to claim 1, wherein after the light irradiation from the light source, sleep pressure, which is expressed as a high level of wakening theta waves, is increased.

8. The PBM system according to claim 7, wherein the increase in sleep pressure creates a brain environment that induces an enhancement of delta waves during sleep.

9. The PBM system according to claim 1, wherein the light irradiation from the light source is performed prior to sleep onset.

10. The PBM system according to claim 1, wherein the control unit adjusts the light wavelength, irradiation intensity, and duration of irradiation.

11. A sleep induction method using the PBM system of claim 1.

12. A sleep induction device employing the PBM system of claim 1, comprising:
a light source unit having at least one LED element;
a control unit which adjusts the output of the light source;
an internal chassis on which a plurality of light sources and control units are installed; and
an external chassis on which a charging cord and connection terminals are installed.
